(19) United States
(12) Patent Application Publication
(10) Pub. No.: US 2018/0015132 A1
(43) Pub. Date: Jan. 18, 2018

(54) METHOD FOR TREATMENT AND PREVENTION OF KIDNEY DISEASES WITH LOTUS SEEDPOD EXTRACT

(71) Applicants: Jing-Hsien Chen, Taichung City (TW); Hui-Hsuan Lin, Taichung City (TW)

(72) Inventors: Jing-Hsien Chen, Taichung City (TW); Hui-Hsuan Lin, Taichung City (TW)

(21) Appl. No.: 15/642,306

(22) Filed: Jul. 5, 2017

(30) Foreign Application Priority Data
Jul. 12, 2016 (TW) 105121949

(51) Int. Cl. A61K 36/48

(52) U.S. Cl. CPC A61K 36/48; A61K 2236/331

(57) ABSTRACT

Provided is a method for treatment and prevention of kidney disease with a composition, wherein the composition comprises a lotus seedpod extract.

### CROSS-REFERENCE TO RELATED PATENT APPLICATION

This application claims priority under 35 U.S.C. §119(a) to Taiwan Patent Application No. 105121949, filed on Jul. 12, 2016, the content of which is hereby incorporated by reference in its entirety.

### BACKGROUND OF THE INVENTION

### 1. Field of the Invention

The present invention relates to a method for treatment and prevention of kidney disease with plant extracts, particularly a method for treatment and prevention of kidney disease with a composition that comprises a lotus seedpod extract.

### 2. Description of the Prior Arts

Kidney is an important metabolic organ of the human body, mainly to filter uric acid, urea nitrogen or creatinine and other substances metabolized by the body, which are formed into urine with the electrolyte and water and then excreted from the body. Kidney damage will make the body gradually lose the normal metabolic function, causing a great threat to health. As the kidney disease has no obvious symptoms in the early stage, most patients with kidney disease would not find that they have been ill until the late stage. At that stage, the kidney disease cannot be cured simply through drug treatment without dialysis or renal transplantation.

According to statistics, the number of patients with kidney disease in the world continues to increase, resulting in a heavy burden of medical resources and expenditure. In addition, according to the Taiwan Ministry of Health and Welfare statistics in 2014, kidney disease is one of the top 10 causes of death in Taiwan, so kidney disease has become an important public health issue. With the recently attached importance to natural healing methods and the biologically active composition in plants, many studies are focused on the development of the efficacy of active compositions extracted from plants.

### SUMMARY OF THE INVENTION

The present invention provides a method for treatment and prevention of kidney disease with a composition that comprises a lotus seedpod extract.

In order to achieve the above and other objectives, the present invention provides a method for treatment and prevention of kidney disease with a composition that comprises a lotus seedpod extract. Administering an effective amount of the lotus seedpod extract to a subject can effectively prevent and/or treat a kidney injury-related disease.

Preferably, the lotus seedpod extract is obtained by water extraction. More preferably, the lotus seedpod extract is obtained by hot water extraction.

Preferably, the kidney disease is renal fibrosis related disease.

Preferably, the kidney disease is renal inflammation related disease.

Preferably, the kidney disease is chronic renal failure.

Preferably, the kidney disease is gouty nephropathy.

Preferably, the effective amount of the lotus seedpod extract administered to a mouse is between 2 mg per g per day and 0.35 mg per g per day.

Preferably, the effective amount of the lotus seedpod extracts administered to human is between 0.15 g per kg per day and 0.35 g per kg per day. The effective dose of the lotus seedpod extracts administered to human is calculated based on the initial experimental method of the US Food and Drug Administration published in 2005: estimating the maximum safe starting dose in initial clinical trials for therapeutics in adult healthy volunteers.

Preferably, the composition further comprises a pharmaceutically acceptable carrier.

The pharmaceutically acceptable carrier in accordance with the present invention includes, but is not limited to, solvent, emulsifier, suspending agent, decomposer, binding agent, excipient, stabilizing agent, chelating agent, diluent, gelling agent, preservative, lubricant, surfactant, and any other similar or applicable carrier of the present invention.

### BRIEF DESCRIPTION OF THE DRAWINGS

Objectives, features, and advantages of the present invention are hereunder illustrated with specific embodiments in conjunction with the accompanying drawings, in which:

FIG. 1 is the representative view of the kidneys of each group;

FIG. 2 is the histogram of the kidney weights of each group;

FIG. 3 shows the expression of TNF-α of each group;

FIG. 4 shows the expression of IL-6 of each group;

FIG. 5 shows the specific activity of glutathione peroxidase of each group;

FIG. 6 shows the specific activity of glutathione reductase of each group;

FIG. 7 shows the glutathione content of each group;

FIG. 8 shows the specific activity of hydroperoxidases of each group;

FIG. 9 shows the specific activity of superoxide dismutase of each group;

FIG. 10 shows the total antioxidant capacity of each group;

FIG. 11 shows the degree of lipid peroxidation of each group;

FIG. 12 shows the mouse kidney slices stained with H & E; and

FIG. 13 shows the mouse kidney slices stained with Masson's trichrome stain.

### DETAILED DESCRIPTION OF THE PREFERRED EMBODIMENTS

Before the present invention is described in greater detail with reference to the accompanying examples, it should be noted herein that like elements are denoted by the same reference numerals throughout the present invention.

The results of all the experimental data are expressed as the mean±standard deviation (mean±SD), where the results of another group compared with the first group are shown with “#” representing p<0.05, and “##” representing p<0.01; the results of another group compared with the second group are shown with “*” representing p<0.05, and “**” representing p<0.01.

### Preparation Example 1: Preparation of the Lotus Seedpod Extract

100 g dried lotus seedpod was added into 4 L distilled water, heated for about 1 hour to 2 hours, and then filtered to obtained a supernatant.

The supernatant was freeze-dried to obtain a powder-like lotus seedpod extract, and then the powder-like lotus seedpod extract was dissolved in the water to prepare samples in different concentrations for subsequent experiments.

### Preparation Example 2: Establishment of Chronic Kidney Injury Animal Models

6-week-old male C57BL/6 mice were obtained from Experimental Animal Center of the National Experimental Institute. Each mouse weighs about 25 g, and was given 5 g of feed daily, and these mice were randomly divided into four groups: the first group as control group; the second group as adenine-induced group; the third group which was induced by adenine was fed with 1 weight percent lotus seedpod extract (of total weight of feed); and the fourth group which was induced by adenine was fed with 2 weight percent lotus seedpod extract (of total weight of feed).

The incubation conditions were as follows: automatic air conditioning (12 times per hour), automatic light control (12 hours light, 12 hours dark), room temperature control at 22±2° C., relative humidity ranging from 50% to 55%, and general feed and water feeding. The experiment lasted for 9 weeks. The mice of the second group to the fourth group were administered 0.2% by weight of adenine of the total weight of the feed daily to induce kidney damage. From the fourth week onwards, the third group and the fourth group of mice were respectively further administered 1-weight percent (2 mg per day) of the total weight of the fed lotus seedpod extract, and 2-weight percent (4 mg per day) of the total weight of the fed lotus seedpod extract. After the experiment was completed, the mice were sacrificed and their kidneys were subjected to biochemical analysis.

### Example 1: Kidney Weight Analysis

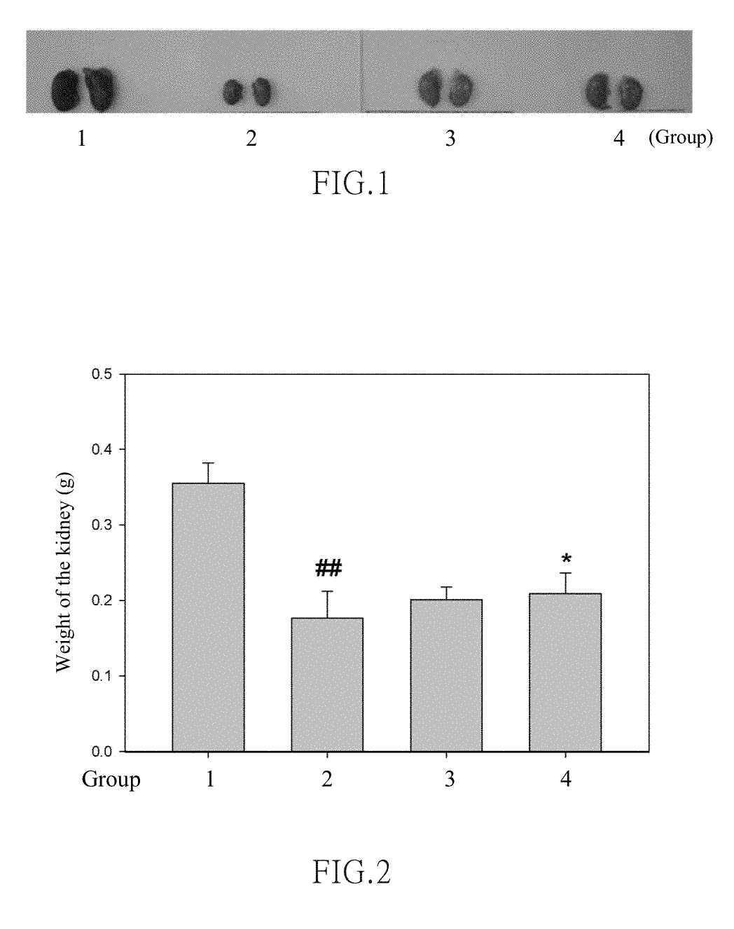

The kidneys of mice of each group were weighed and photographed to obtain FIGS. 1 and 2.

Compared to the first group, the kidneys of the second group were obviously atrophied and the weight of the kidneys was lighter. These results show that administration of adenine causes kidney damage. Compared to the second group, the kidneys of the third group and the fourth group gained weight and were only slightly atrophied, and the weight of the kidneys was significantly maintained as the concentration of lotus seedpod extract increased. The results show that the lotus seedpod extract is effective for improving and treating kidney damage and its associated diseases.

### Example 2: Biochemical Analysis of Blood

Blood collected by cardiac puncture was centrifuged at 3000 rpm to obtain the supernatant for biochemical analysis, and the results were shown in Table 1 below.

TABLE 1
Blood biochemical analysis of each group of mice

| Group | 1 | 2 | 3 | 4 |
|---|---|---|---|---|
| Blood urea | 33.35 ± | 130.16 ± | 106.28 ± | 110.32 ± |
| nitrogen | 1.82 | 17.22^{##} | 7.79* | 7.71* |
| (mg/dl) | | | | |
| Creatinine | 0.35 ± | 0.92 ± | 0.73 ± | 0.65 ± |
| (mg/dl) | 0.05 | 0.15^{##} | 0.05* | 0.06* |

Table 1 shows that the blood urea nitrogen and creatinine of the second group were respectively 3.90 times and 2.63 times of the first group. Compared with the second group, the blood urea nitrogen in the third group and the fourth groups were decreased by 0.82 times and 0.84 times, and the creatinine values were decreased by 0.79 times and 0.71 times, respectively. These results show that the lotus seedpod extract can effectively improve renal dysfunction, so as to achieve treating or improving kidney damage-related diseases.

### Example 3: Inflammatory Factors Analysis of Blood

The expression of IL-6 and TNF-α in the blood of each group were detected by enzyme-linked immunosorbent assay (ELISA assay). This example was analyzed by using the BioLegend ELISA MAX™ Deluxe Sets, and the detailed experimental steps are within the general knowledge of the art to which this invention pertains and are not intended to be a technical feature of the present invention, and are therefore not described here.

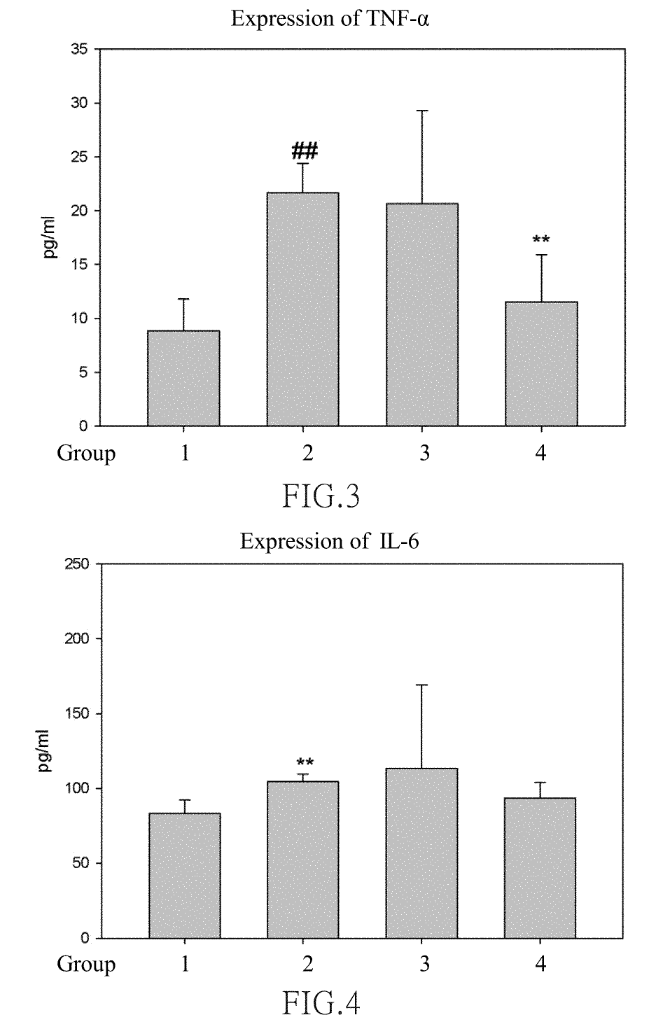

According to the results shown in FIGS. 3 and 4, TNF-α and IL-6 expressions of the second group were respectively 1.95 times and 1.25 times of the first group, which indicates that adenine will cause renal inflammation and then lead to kidney diseases. TNF-α expressions of the third group and the fourth group were respectively 0.97 times and 0.76 times of the second group, and IL-6 expressions of the third group and the fourth group were respectively 1.08 times and 0.89 times of the second group. It was found that the lotus seedpod extract of the present invention can improve or treat renal inflammation or its associated diseases at high doses.

### Example 4: Analysis of Antioxidant Enzyme Activity

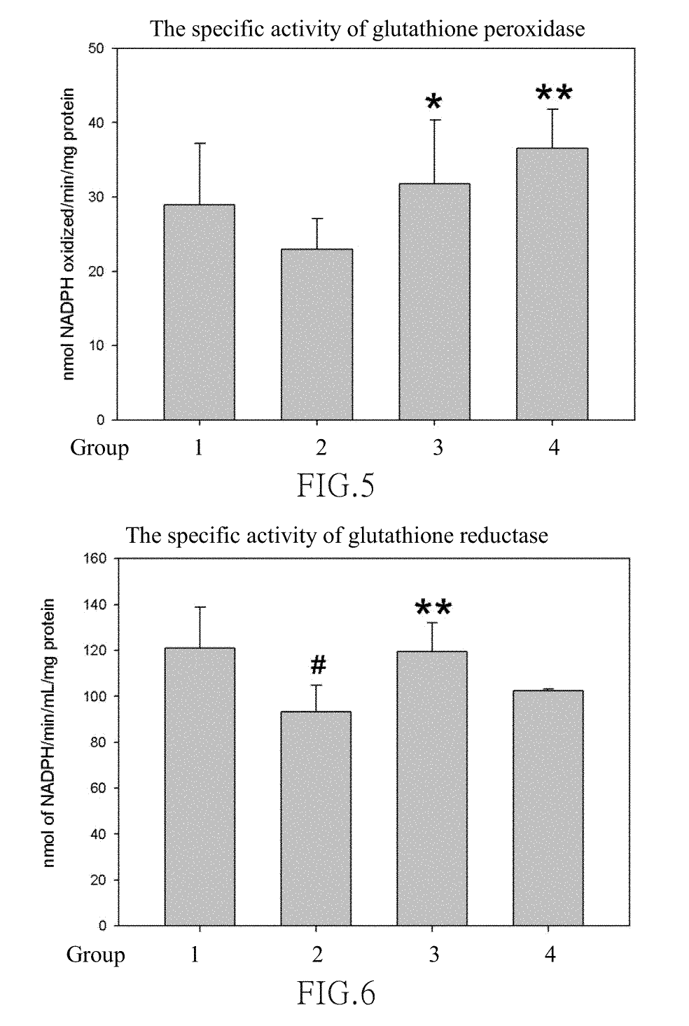

The renal tissues of each group were collected for analysis of antioxidant enzyme activity. The antioxidant enzyme activities include glutathione peroxidase activity, glutathione reductase activity, glutathione content, hydroperoxidases activity, and superoxide dismutase activity, the total antioxidant capacity and the degree of lipid peroxidation. These results were shown in FIGS. 5 to 11. These test methods are within the general knowledge of the art to which this invention pertains and are not intended to be a technical feature of the present invention, and are therefore not described here.

Referring to FIG. 5, the specific activity of glutathione peroxidase of the second group was 0.79 times of the first group. The specific activities of glutathione peroxidase of the third group and the fourth group were respectively 1.38 times and 1.59 times of the second group. Referring to FIG. 6, the specific activity of glutathione reductase of the second group was 0.77 times of the first group. The specific activities of glutathione reductase of the third group and the fourth group were respectively 1.28 times and 1.09 times of the second group.

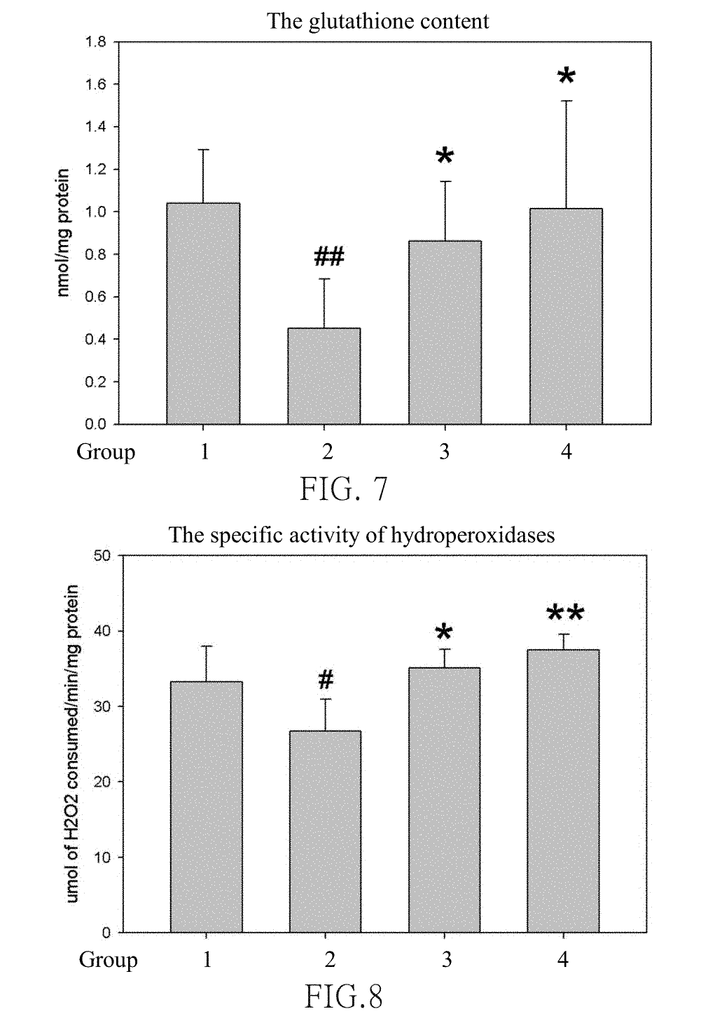

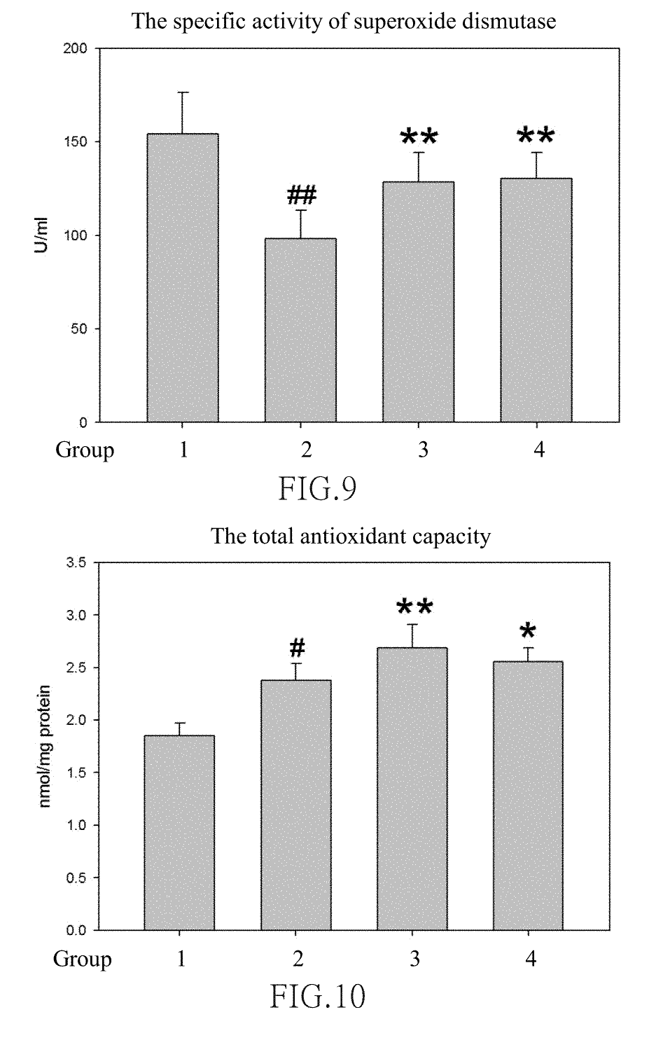

Referring to FIG. 7, the glutathione content of the second group was 0.43 times of the first group. Compared to the second group, the glutathione content of the third group and the fourth group were both increased, 1.91 times and 2.25 times of the second group, respectively. Referring to FIG. 8, the specific activity of hydroperoxidases of the second group was 0.79 times of the first group, while the specific activities of hydroperoxidases of the third group and the fourth group were respectively 1.32 times and 1.42 times of the second group. Further referring to FIG. 9, the specific activity of superoxide dismutase of the second group was 0.62 times of the first group. Compared to the second group, the specific activity of superoxide dismutase of the third group and the fourth group were both obviously increased, 1.33 times and 1.35 times of the second group, respectively.

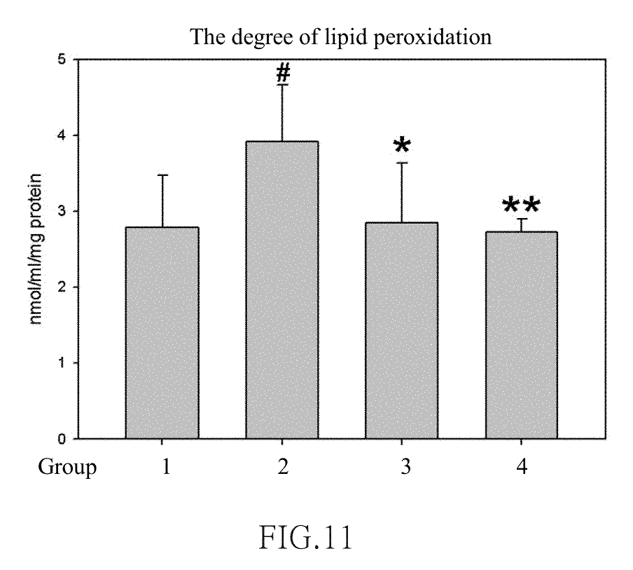

Referring to FIG. 10, the total antioxidant capacity of the second group was 1.27 times of the first group, and compared to the second group, the total antioxidant capacity of the third group and the fourth group were both increased, 1.13 times and 1.07 times of the second group, respectively. Referring to FIG. 11, the degree of lipid peroxidation of the second group was 1.4 times of the first group, while the degree of lipid peroxidation of the third group and the fourth group were respectively 0.72 times and 0.69 times of the second group.

The above results found that the administration of the lotus seedpod extract to the chronic kidney injury animal models can increase the specific activities of the glutathione peroxidase, glutathione reductase, hydroperoxidase and superoxide dismutase, glutathione content, and also reduce the degree of lipid peroxidation. In other words, the lotus seedpod extract of the present invention is capable of reducing the oxidative pressure by enhancing the activity of the antioxidant enzymes, so as to achieve the renal protection, and renal injury treatment or improvement.

### Example 5: Histochemical Staining

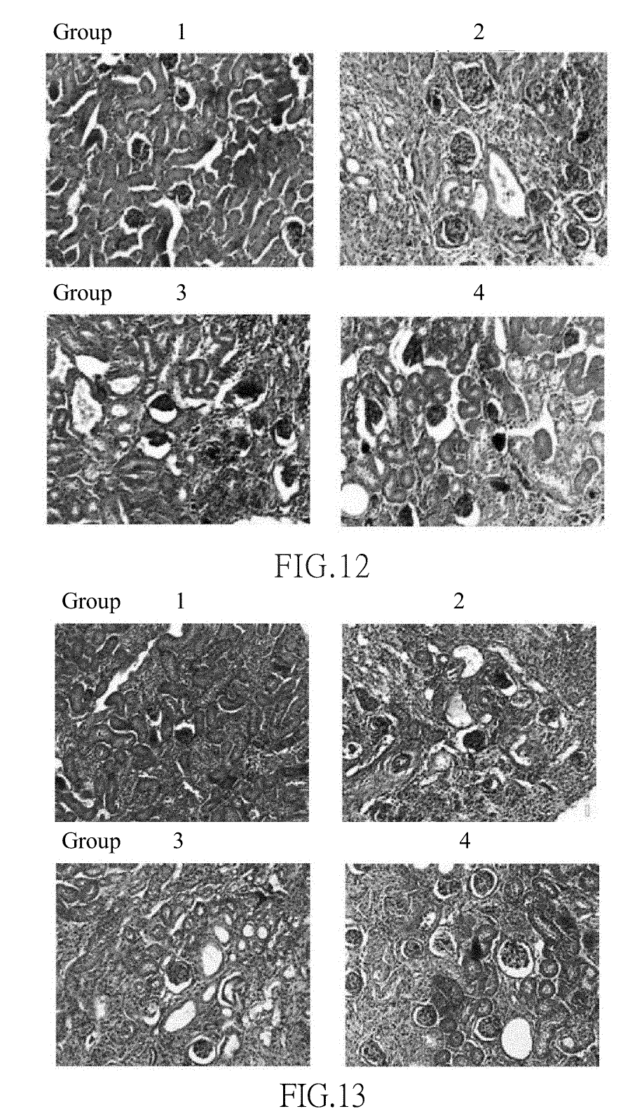

The kidneys of each group were paraffin-embedded and sliced into 5 μm thickness. The samples were respectively stained with H & E and Masson's trichrome stain. The results were shown in FIG. 12 to FIG. 13.

Referring to FIG. 12, the proximal convoluted tubules and distal convoluted tubules around the glomerulus partially had cavitations. As shown in the slices of the third group and the fourth group, the partial cavitations of the proximal convoluted tubules and distal convoluted tubules around the glomerulus were less serious compared with that of the second group, and the overall color and tissue type were closer to the first group, wherein the tissue type of the fourth group was closer to the first group than the second group.

Referring to FIG. 13, the degree of renal fibrosis of the second group was more obvious than the first group and the degree of renal fibrosis of the third group and the fourth group, which were administered with the lotus seedpod extract, were less obvious compared to the second group.

These results show that administration of the lotus seedpod extract of the present invention to renal injury model can effectively protect the kidneys and reduce renal fibrosis. In addition, the higher the concentration of the lotus seedpod extract, the better the efficacy on kidney damage improvement. Thus, the lotus seedpod extract of the present invention can achieve the efficacy of treating and/or preventing renal damage or renal disease.

The lotus seedpod extract of the present invention can be used as an active composition for the treatment and/or prevention of kidney disease or its associated diseases, as has been confirmed by the examples described above. Therefore, it is possible to prepare a pharmaceutical composition or a food product in accordance with commercial needs with a pharmaceutically acceptable carrier or a food-acceptable ingredient. Further, the lotus seedpod extract of the present invention is an edible plant as a raw material, and water is used as an extraction solvent, and thus it can be used safely in the human body.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for treatment and prevention of kidney disease with a composition, wherein the composition comprises a lotus seedpod extract.

2. The method according to claim 1, wherein the lotus seedpod extract is obtained by water extraction.

3. The method according to claim 1, wherein the kidney disease is renal fibrosis related disease.

4. The method according to claim 1, wherein the kidney disease is renal inflammation related disease.

5. The method according to claim 1, wherein the kidney disease is chronic renal failure.

6. The method according to claim 1, wherein the kidney disease is gouty nephropathy.

7. The method according to claim 1, wherein the effective amount of the lotus seedpod extract is between 0.15 g per kg per day and 0.35 g per kg per day.

8. The method according to claim 1, wherein the composition comprises a pharmaceutically acceptable carrier.

9. The method according to claim 2, wherein the composition comprises a pharmaceutically acceptable carrier.

10. The method according to claim 3, wherein the composition comprises a pharmaceutically acceptable carrier.

11. The method according to claim 4, wherein the composition comprises a pharmaceutically acceptable carrier.

12. The method according to claim 5, wherein the composition comprises a pharmaceutically acceptable carrier.

13. The method according to claim 6, wherein the composition comprises a pharmaceutically acceptable carrier.

14. The method according to claim 7, wherein the composition comprises a pharmaceutically acceptable carrier.
